(19) United States
(12) Patent Application Publication
(10) Pub. No.: US 2022/0118126 A1
(43) Pub. Date: Apr. 21, 2022

(54) Anti-Microbial Hand Sanitizing Device

(71) Applicant: Richard D. Yanke, Burbank, CA (US)

(72) Inventor: Richard D. Yanke, Burbank, CA (US)

(73) Assignee: Richard D. Yanke, Burbank, CA (US)

(21) Appl. No.: 17/501,639

(22) Filed: Oct. 14, 2021

Related U.S. Application Data
(60) Provisional application No. 63/092,253, filed on Oct. 15, 2020.

(51) Int. Cl. A61L 2/00; A61L 2/24; A45D 29/00

(52) U.S. Cl. CPC A61L 2/0047; A61L 2/24; A61L 2202/14; A61L 2202/11; A61L 2202/122; A45D 29/00

(57) ABSTRACT

The invention relates to a system, methods and apparatus for efficiently and cost-effectively preventing the transfer of microbes, such as, for example, bacteria and viruses through hand contact, typically encountered in a retail store, industrial setting or healthcare facility or other public area. The invention further considers the application of the methods and apparatus in other industrial disciplines where subjects may be susceptible to cross-contamination of microbes by hand contact. One embodiment incorporates a box where the user simple inserts their hands into a suitable opening. Especially designed lamps allow for a rapid switching means to control the time period for activating or deactivating lamps, thus regulating UV exposure. Exposure time with UVC and the orientation of the UV lamps provides complete germicidal decontamination within seconds.

### RELATED APPLICATIONS

This application claims the benefit of U.S. Provisional Patent Application No. 63/092,253, filed Oct. 15, 2020, the disclosure of which is incorporated herein by reference.

### FIELDS OF THE INVENTION

The invention presented here relates to an ultraviolet sterilization system used for preventing the transfer of microbes on a subject's hand, specifically the present invention relates to light treatments applied to human skin tissue and in particular to light treatments utilized within a confined irradiation chamber. The design can be easily used by the general public to rapidly inactivate bacteria, viruses, fungi, prions, viroids and spores along the skin surface of the subject's hand.

### BACKGROUND

Unwanted and dangerous microbes can be found on skin surfaces of most subjects having normal hand use or interaction with contact surfaces. Contact with microbes have been associated with community interactions in public forums such as hospitals, work environments, commercial buildings where acquired infections through hand touching can further account for a substantial yearly expense to insurance companies. This can also include equipment or components found in water processing plants, food processing plants, dairies, livestock habitation facilities, retail stores, etc., all susceptible to bacteria growth.

One method of reducing the spread of germs and diseases through hand contact is to use proper hand care and sanitization. This is especially true in industries or areas where bacterial contamination is particularly problematic, such as health care and food and beverage industries. Further relevant is in a commercial retail situation where the general public is asked to practice safe hygiene.

Hand washing with soap and water is commonly used, but hand sanitizers that do not need to be washed off have become popular as well. Typically, a subject applies hand sanitizer onto the hands and spreads it around such that the hand sanitizer contacts most or all of the skin on the hands. These sanitizers are available in gels or foams, and many include antimicrobial agents which can be found in hospitals and other healthcare environments, the workplace, retail stores, or in everyday home use. While these methods provide some level of protection, some illnesses associated with microbes have been known to survive exposure to alcohol.

One advantage of using ultraviolet (UV) light to kill bacteria is that it is not susceptible to the danger of antimicrobial resistance that can occur without the use of pharmacologic or chemical agents. This can avoid potential side effects associated with many pharmacologic or chemical agents.

Accordingly, it is desirable to provide a means to hand sanitize with an enhanced antimicrobial efficacy without the need to provide chemicals or other mechanical cleaning procedures in a low-cost, efficient, and rapid antimicrobial protection process for a human subject. Other desirable features and characteristics of the present embodiment will become apparent from the subsequent detailed description and the appended claims, taken in conjunction with the accompanying drawings and this background of the invention.

### SUMMARY

Individual subject's hand to hand or hand to inert object surface creates physical contact that, in part, can spread known microbes or diseases to multiple people and consequently become an inadvertent vector for the transmission of microbes in communities. For example, this can be especially true in large retail stores where patrons select their purchase items using their hands only to infect themselves or further spread the problem. Thus, the risk of communicating a plethora of microbial organisms increases with continued contact. Subjects, especially people with suppressed immune systems, are unnecessarily exposed to multiple diseases.

The present invention incorporates an ultraviolet sanitization system that can inexpensively, safely and efficiently irradiate a person's hand with ultraviolet light (UVC). In one embodiment, an enclosed chamber having, an outer casing configured to substantially enclose a person's hand or hands into an inner area within the enclosed chamber is configured to receive each hand, and a series of electrical components designed to completely irradiate with ultraviolet light, specifically UVC, the surface area of the hands are optimized for a time interval that allows for a uniform and complete removal of microbes on the hand surface.

The present invention offers a type of personal protective equipment (PPE) with germicidal protection in a low-cost, rapid, and efficient system. This “real time” UV irradiation make the present invention useful in most retail stores and large commercial entities or in any situation where the public or a group of people may be at risk to hand contact.

The embodiments of the present invention are shown in the drawings and summarized below. It is to be understood, however, that there is no intention to limit the invention to the forms described in the specification. One skilled in the art can recognize that there are numerous modifications that would embody the spirit and scope of the invention as expressed in the claims.

### DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 Images of irradiation chamber design having a single chamber area with internal hand positioning area. Panel A is a top view of the irradiation chamber containing the inner chamber irradiation area. Panel B is a front face view with two hand access openings for hand insertion.

FIG. 2 A perspective view showing the outer front face of the irradiation chamber with light protective holes for hand placement and indicator led placement. Shown is a single hand positioned for inserting into right hole.

FIG. 3 Block diagram of main control board.

FIG. 4 Image of a micro switch design to accurately time the signaling of the UVC lamp.

FIG. 5 Image of the recessed metal pocket housing the 12 volt power connection as well as the POS interface.

FIG. 6 Image of the ultraviolet-C board and lamp design.

FIG. 7 Images of the modular design of the PCB lamp circuit board assembly. Panel A shows the UV-C circuit board and lamps mounted on an insulated platform. As shown in Panel B, a staggered pin orientation provides for simple installation.

FIG. 8 Panel A shows an image of the protective screen inserted into position along the front and back 45 degree angle screen and platform edge. Panel B shows the modular circuit board and lamp with protective screen assembled on the inner surface of the top of the outer box. Panel C show an alternative design with standoff pins.

FIG. 9 Image of the aluminum protective screen shown on inner surface of the top side of the currency drawer.

FIG. 10 Insider view of the aluminum protective screen showing sides angled at 90 degrees with portions cut open for wire access and on the front and back edges a 45 degree angle.

FIG. 11 Top view of a kiosk design used in retail having outside box with a front face and access holes on stand alone unit.

FIG. 12 Panels A and C are images of two petri dishes sampled from currency obtained from a restaurant and pharmacy, respectively. Each petri dish was inoculated from 3 different bills used in a typical transaction within the store and without exposing to the UV-C cash drawer system of the present invention. Panels B and D are images of petri dishes sampled from the same currency in Panel A and C, respectively, after disinfection using the UV-C in the cash drawer. Samples for each petri dish were obtained under aseptic conditions with each dish incubated for 72 hours at 32° C.

### DETAILED DESCRIPTION OF THE INVENTION

UV lamps have been described for several portable devices as a sterilization means. For example U.S. Pat. No. 4,786,812 issued on Nov. 22, 1988 describes a hand-held device to sterilize a surface contaminated with mold, yeast or virus using ultraviolet (UV) lamps operating at a wavelength of 253.7 nanometers. U.S. Pat. No. 4,896,042 issued on Jan. 23, 1990 describes a two-piece device consisting of a hand-held unit with UV lamps for sterilization of surfaces and a base unit with a fan onto which the hand-held unit is secured for the sterilization of the surrounding atmosphere. Neither invention provides for or can ensure the complete decontamination of a subjects hands prior to personal use.

None of the prior art inventions provide for or can safely and efficiently ensure complete decontamination of a subjects hands prior to routine use in a place such as a public area. These sanitization in a device that does not require chemical and/or mechanical long exposure times and cumbersome manipulation which delays any quick and efficient process for decontamination.

The device described in the present invention provides a system for significantly reducing or eliminating the presence of microbes found on the surface of a user's hands. One embodiment incorporates an enclosed chamber or box used within a public area or a large commercial entity such as in retail stores. The irradiation chamber is equipped internally with a series of ultraviolet lamps (UVC) programed to irradiate a person's hand upon placement within the box. UVC bulbs and electronic circuitry provide an efficient and commercially practical means to providing a germicidal sanitizer. The irradiation chamber offers an opening for hand placement with shielded access. When the opening is triggered upon hand placement, a calculated dose of germicidal UVC energy is delivered automatically within the box, eliminating or significantly reducing the microbes. LED displays on the outer front face of the box provide the user with the system's status, including power, lamps, and lamp use. Other embodiments of the present invention include, but not limited to, use as a kiosk station in the workplace such as in the healthcare industry or in the food preparatory industry where rapid sanitizing eliminates the need for chemical hand cleaning.

Other areas include applications in public places such as in retail where groups of people require rapid hand sanitization before continued hand contact or other potential exposure risks.

### Overall UVC Germicidal and Antimicrobial System

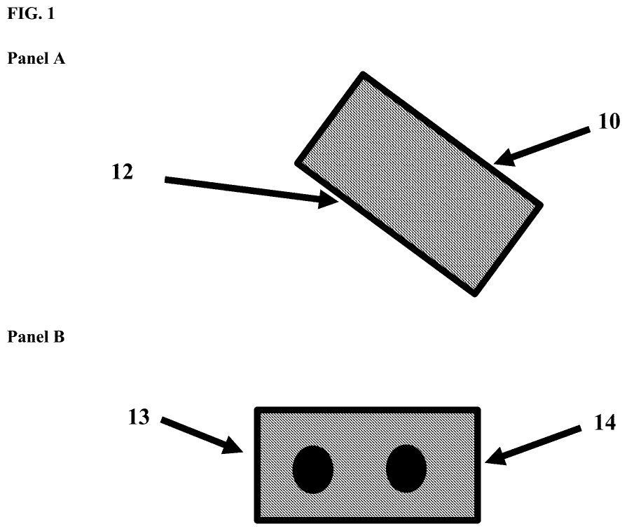

The PCB power circuit board and lamp circuit board is designed to fit in any type of sanitization system to sanitize one or more hands or the surface to be exposed. The target or hand to be irradiated is contained within a closed unit. Typical contained units are shown in FIG. 1 as a box. FIG. 1 Panel A is a top outside view of the irradiation chamber where an area within the closed box provides easy hand access for placement within the box. As illustrated, FIG. 1 depicts the hand sanitization unit with an outer box frame (10) having a front face to access an inner box (12). While any design for inner area is contemplated in the present invention, FIG. 1 Panel B depicts the front face (14) having a specific access openings as two shielded hole openings (13) for inserting hands prior to irradiating. Upon inserting, the system is activated and the contents are exposed to a UVC source for a predetermined time period optimized to ensure complete elimination of any microbial activity on the surface of the target with the least amount of lamp usage.

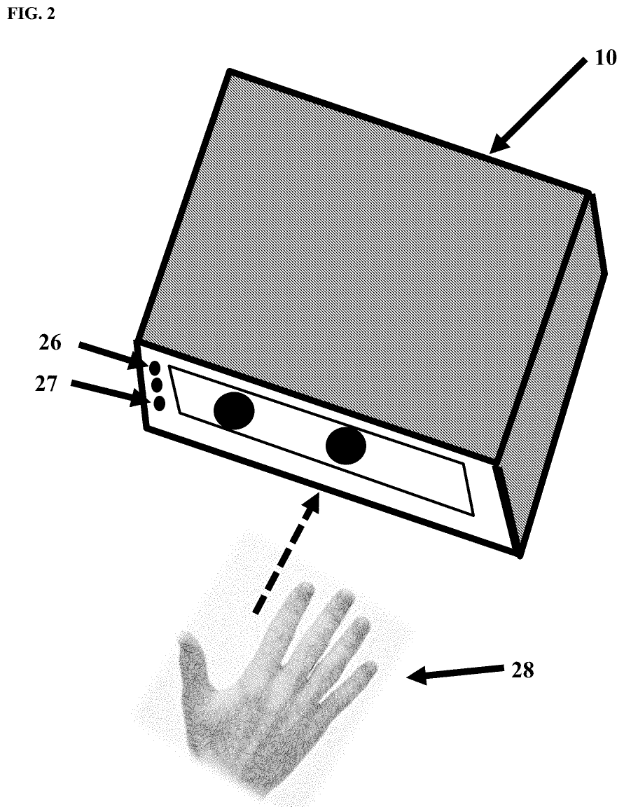

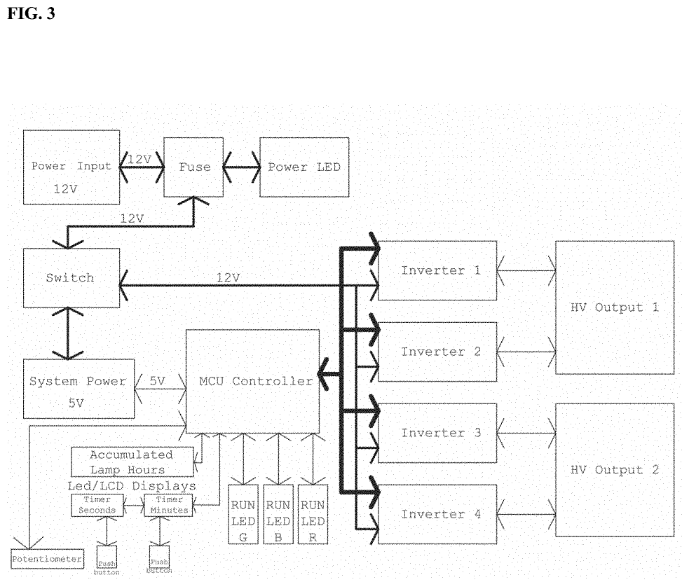

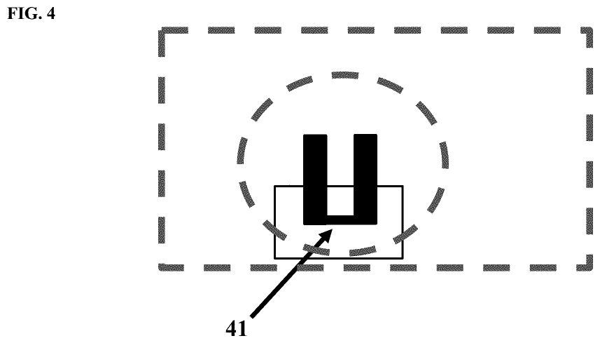

One embodiment of the present invention addresses the need for removing microbial activity on the surface of any items such as jewelry or other items that may be located on the user's hands. The present invention utilizes, in part, the standard design of a box (10) having an access opening but also incorporating a UVC irradiation system as shown in FIG. 1. FIG. 2 further details components on the left side of the front outer face of the box are green (26) and blue (27) LED indicator lights powered directly from the circuit board described herein. Alternatively, a third red LED indicator light illuminates when the clock on the circuit board reaches 10,000 hours which functions to notify the user that the bulb is to be replaced. While the present invention considers both simultaneous insertion of a subject's left and right hands, FIG. 2 depicts the upper top portion of a subject's single right hand (28) and nail placement prior to insertion into the right hole (see dashed arrow). FIG. 3 provides a block diagram of the main control board, showing the interrelationships between the irradiation lamps and user indication LEDs. FIG. 3 incorporates the third red LED which alternatively can be used to assess the life of the UV bulbs.

Inside the outer box, a micro sensor triggers the germicidal system through a specially designed connector (41). Shown is the front face as dashes incorporating one shielded hand access hole. This insures smooth operation and accuracy of the timing which corresponds to the hand placement in the germicidal chamber or by any other means in overall UVC operation.

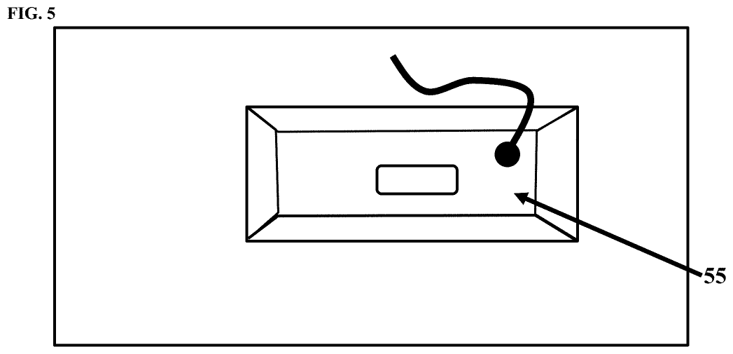

Optionally, the outside bottom of the outer box in FIG. 5 has a 2¾″×1½″×2″ deep metal pocket (55) modified to contain a 12 volt power connection as well as the box POS interface. By recessing this pocket and locating the connectors within the pocket, the connectors provide protection and the drawer will sit flat.

Special quick release power plugs and connectors on the sterilization system as well an easily removable main PCB microprocessor circuit board and a PCB Ultraviolet-C lamp circuit board facilitate the efficient exchange of the parts of these components, allowing for cost-effective, quick and efficient use on site. The quick release plugs and connectors on the box allow for rapid assembly and field repair.

The present invention considers all possible applications of the general embodiment of the present invention. Not only can it be applied to a self-contained, stand-alone box, but placement of the box has applications in access to a public building where a kiosk may be used to check a person's temperature while they are irradiating their hands. When used in conjunction with an on-screen video, the box may provide a source of entertainment for the user while their hands are sanitized.

Another application is in the food industry where rapid efficient access to a hand irradiation chamber is important. The box is placed near the work area and users are able to quickly irradiate their hands before or after working. An optimized exposure time to UVC irradiation allows complete sanitization in-situ. Typical application would involve sanitization by simply having the subject place their hands in the box and exposing to UVC irradiation for a predetermined time period.

### UVC—PCB Circuit Board Germicidial Sterilizer

FIG. 3 shows a block diagram of the main control board. All components are UL certified or are UL recognized components. A 2 pin male connector links the 12 volt DC input with a 3 amp fuse on circuit board. Another 2-Pin male connects to 2 pin female connector attached to 18 gauge+-wires that connect to a 2.1 mm barrel power connector that installs in the bottom of the unit in the recessed pocket. The unit further contains (4) AC/DC high voltage rms transformers.

A microcomputer processor mounted on shock absorbing rubber grommets is incorporated to provide a custom program and a micro switch which functions in the assimilation of a one-shot timer, controlling the transformers and LEDs. The blue LED UVC indicator light and the UVC germicidal lamps are switched on or off to indicate the status of the power or UV light, respectively. The processor also controls a potentiometer to supply the time required for the antimicrobial process. It is used to monitor, store and display the information and is set by two separate minute and second push button digital micro switches which when pressed will display the time that the UVC lamps will remain active, resulting in a much improved accuracy in regulating the amount of time the UV light is on, allowing for improved optimization of irradiation time intervals, and in standardizing the manufacture/product of multiple devices for a specific application. The processor is also used to monitor, store and display the information of the total accumulated time the UVC lamps have been active, thus providing an indication for replacement. The processor is responsible for monitoring and sending information to the red LED light when the life of the lamps has been exhausted and notifying the user to replace lamps. The LED green light on the front outside face of sterilization unit is constantly illuminated when power is applied to the unit. LED blue light on front outside face of the hand sanitization unit is illuminated only when power is applied from the micro switch, causing the microcomputer processor to engage the AC inverter to power the UVC bulbs and engage the timer, potentiometer and the LCD or LED readout. The LED (or LCD) panel on the outer front face of circuit board is controlled by the microcomputer processor which sends commands to the LED or LCD panel to display the accumulated time the UVC bulbs have been on. On the main PCB circuit board there are 2 brown male 11 pin output connectors. These connectors receive a set of custom designed 5000 k AC voltage silicone wires consisting of 4 red power and one white ground for AC voltage. Each set of cables has a brown female 11 pin connector on one end that connect to the circuit board and a white female 11 pin connector that connects to the Ultraviolet-C lamp circuit board.

### The PCB Ultraviolet—C Lamp Circuit Board

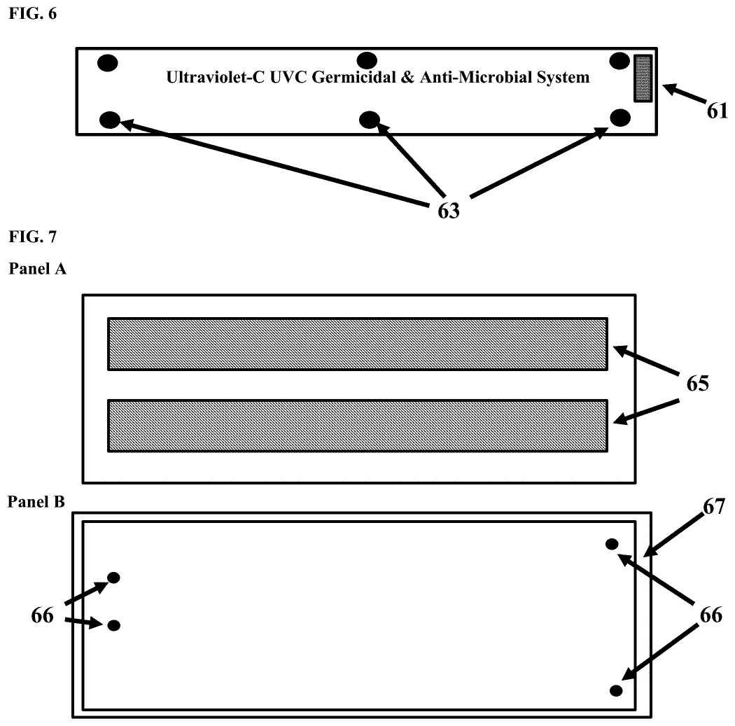

The PCB Ultraviolet-C (UVC) lamp and circuit board is shown in FIG. 6. The board is 14 inch by 2⅞ inch with one white 11 pin male connector input (61). The 11 pin male connector is soldered to four traces that are designed for two germicidal UVC lamps, 5 watt, (5 mm×240 mm) having 253.7 nm wavelength with 2600 microwatts at 1 inch per cm². The lamps are staggered 2″ left to right on the Ultraviolet-C lamp circuit board to provide complete coverage of the target area. These lamps are also soldered onto the Ultraviolet-C lamp circuit board so as to maintain a secure connection throughout their use. The lamps are designed to start quickly by using 1500 vrms to start and 900 vrms to run. This combination of power, size and characteristics of the lamp provides a unique feature to the lamps in the present invention, allowing them to start and reach its full power potential while killing 99.999% of germs within seconds instead of the minutes needed in the prior art. Thus, the lamp allows faster service treatment on/off times when used and ensures complete irradiation during routine on/off use.

The PCB lamp circuit board has 3 holes uniformly on each side of board (63). These holes allow a threaded brass insert attached to a steel pin which accompanies a rubber grommet to allow for shock absorption and for the circuit board to float above the attachment surface. A further embodiment of the PCB Lamp circuit board is a ⅜″×12″ strip of hook and loop Velcro on back of the length of board making board simple to exchange when lamps need to be replaced.

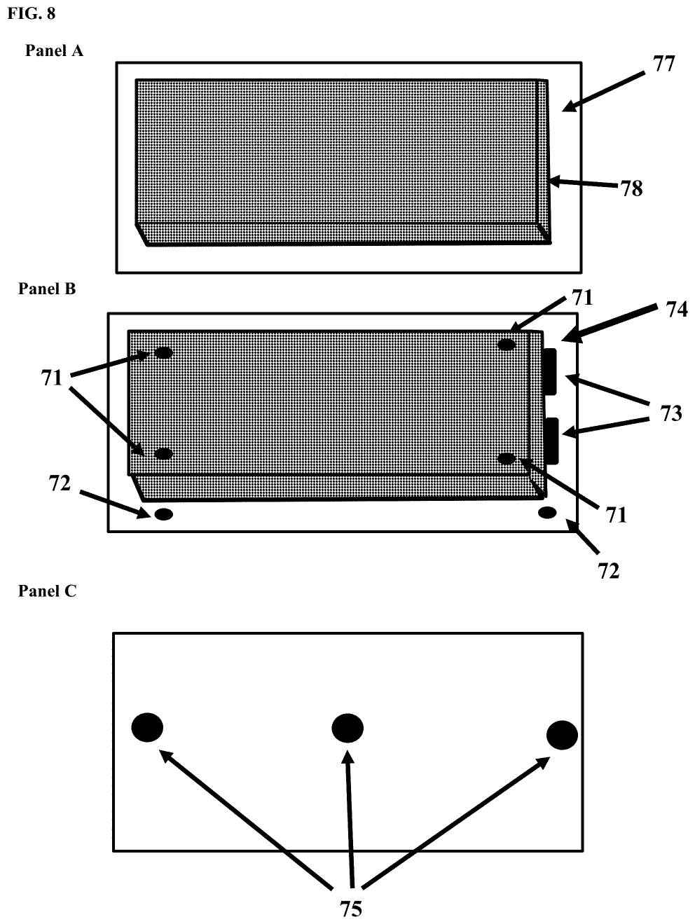

A further embodiment of the PCB Ultraviolet-C (UVC) lamp and circuit board is shown in FIG. 7. FIG. 7 Panel A shows a modular UVC lamp unit for assembly into a UV cash box. Two separate PCB lamp circuit board assemblies (65), each having two UV-C lamps are mounted on the circuit board. The PCB lamp circuit board assemblies (65) are easily mounted on an aluminum platform (67) having an insulated surface and using a staggered pin orientation (66) as shown in Panel B. An aluminum perforated protective screen (78) as described below covers the Ultraviolet-C (UVC) lamp and circuit board. The protective screen is inserted onto the aluminum plate (77) along the front and back 45 degree angle screen edge and a corresponding 45 degree angle edge of the aluminum platform as shown in FIG. 8 Panel A. Once assembled the modular lamp component is quickly and easily installed with 4 screws (71) onto a support platform (72) on the inner top surface of the outer box. The circuit board and lamp are connected to the unit through a two circuit board connectors (73) having a hold-down shield (74) as shown in FIG. 8 Panel B. Panel C depicts another embodiment which incorporates the modular design with standoff pins (75) in the center of the aluminum platform (67) to support the screen and allow for it to be screwed down.

### The Aluminum Perforated Protective Screen

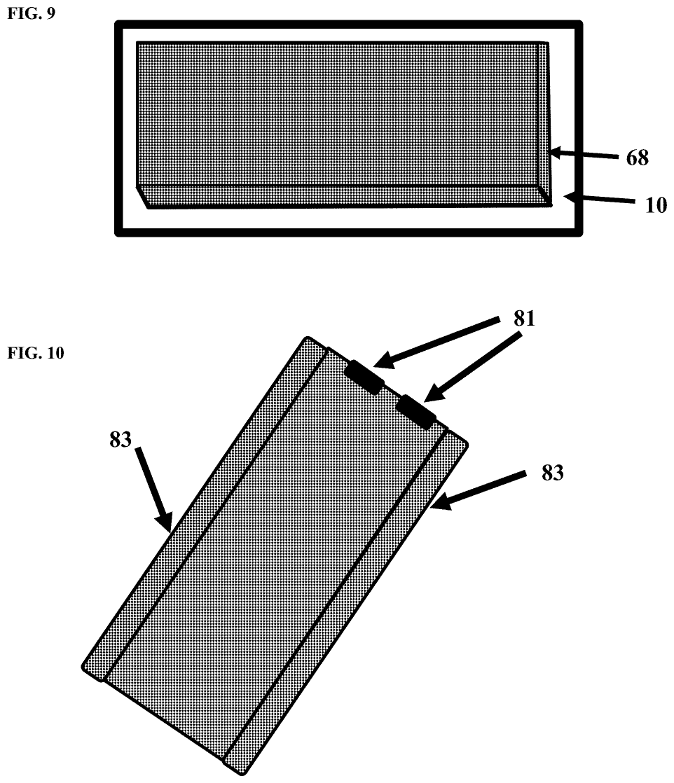

As shown in FIG. 9, a 15″×8″ an aluminum perforated protective screen protects the boards from damage and allows the target, such as coins or paper notes, to be exposed to the generated radiation. FIG. 9 shows the outer box with the bottom side up and with the drawer removed. The aluminum perforated screen (68) is attached to the inner surface of the top side of the outer box (10). As shown in FIG. 10, the screen is angled at 90 degrees on each side with portions cut open for wire access (81) and on the front and back edges up, at a 45 degree angle so as to raise the center of the screen to provide a protective area for the PCB Lamp circuit board. The 45 degree angle also provides deflection for the drawer if it happens to be raised or the system is abused. The system is capable of preventing a high degree of abuse and will not allow the bulbs to break. If the bulbs do break, the screen will provide containment and will not allow the broken lamp to fall into the drawer. The perforation on the screen allows the 254.7 nm light waves to penetrate through and be effective in sterilizing the contents of the drawer. In addition, the screen is painted black on the back side as to not allow any reflection of the light wave so the full strength of the light wave moves through the perforated protective screen.

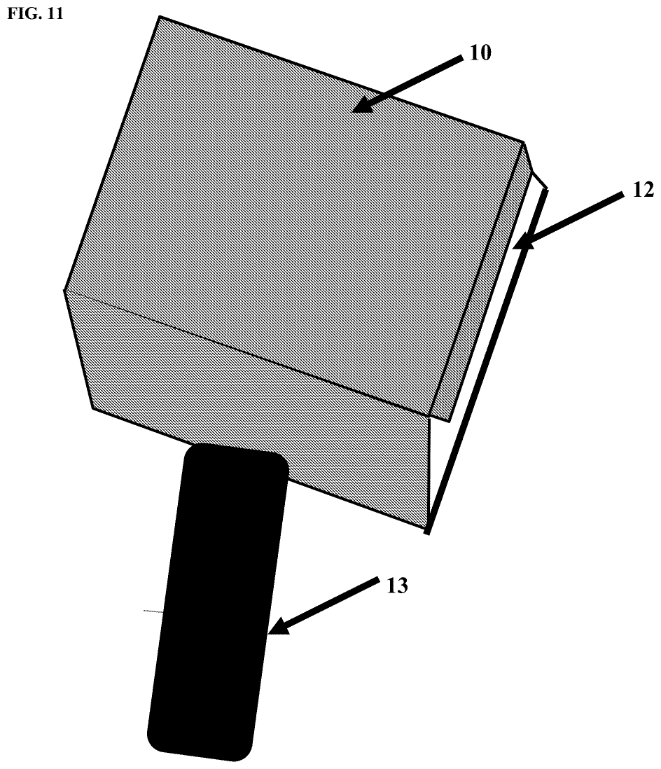

As discussed above, the present invention has applications as a kiosk in retail (see FIG. 11). One embodiment is the use of the outside box (10) and front face with access holes (not shown) (12) as a stand alone unit (13) for public use by individual subjects. Specially designed compartments on the kiosk enable complete exposure of the surface for a single hand or both hanks to the UV-C radiation, thereby ensuring uniform sanitization. To this end, compartments allow light to be reflected from all interior surfaces while the hands are inside the box and the system is activated. A mesh platform (95) sits slightly above the bottom surface of the compartment. The mesh pattern is sufficiently porous as to allow UV-C light to reflect of the bottom drawer surface to irradiate the underside of the tool. The box and kiosk can be further modified to include a functioning video monitor or other appropriate modifications as the irradiation process proceeds.

A still further embodiment of the present invention incorporates a chamber having an outer casing configured to substantially enclose a person's hand or hands into an inner area within the enclosed chamber configured to receive each hand, and a series of electrical components designed to completely irradiate with ultraviolet light, specifically UV-A around approximately 350 nm to allow complete curing on the nail surface of a cosmetically treated finger nail. The surface area of the nails are optimized for a time interval that allows for a uniform and complete cross-linking of acrylic or gel nails. The UV lamps or UV-LED lamps are Ultraviolet-A (UV-A) which provide complete irradiation of acrylics and other monomers, oligomers, or polymers used to create nail enhancement products, including UV gel nails and acrylic liquid and powder nails.

Accordingly, a series of test were performed to demonstrate complete irradiation within the chamber design and support the ability to affect microbes that are needed to disinfect the surface.

### Sample Test

In order to further confirm the ability of the irradiation chamber design to completely affect microbes and disinfect the surface of any currency transferred during the completion of a purchase or at the check-out station, three separate bills were randomly tested. Each selected bill was aseptically stored and transferred by sterile techniques prior to testing. Testing was completed by independently swabbing the surface of the collected bills before and after exposure to the UV-C irradiation chamber design described in the present invention. Petri dishes were incubated for 72 hours at 32° C. and then assessed for microbial growth on the agar.

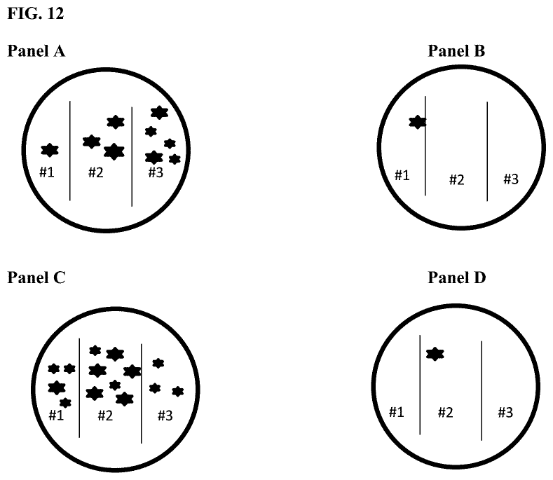

In the first experiment (FIG. 12, Panel A and B), currency exchanged from a fast-food restaurant was selected. Three bills were each independently swabbed to inoculate a sterile petri dish in three separate regions as shown. Panel A depicts growth in each region after 72 hours of incubation. While each bill had varying amounts of microbial growth, Panel A shows the presence of microbes growing on the petri dish in each region, representing their presence on the surface of the sampled bills. In Panel B, the same currency has now been disinfected using the UV-C irradiation chamber design of the present invention. Here, microbe growth has been significantly attenuated, if not completely inhibited, after exposure of the bills to the UV-C irradiation chamber design.

In the second experiment (FIG. 12, Panel C and D), currency exchanged from a pharmacy was selected. Again, three bills were each independently swabbed to inoculate a sterile petri dish in three separate regions. Panel C shows a significantly greater amount of infection in all three bills compared to the restaurant sample, but especially noteworthy is Panel D where the growth is again completely inhibited on all three samples after exposure to the UV-C irradiation chamber design.

The contents of the articles, patents, and patents applications and all other documents and electronically available information mentioned or cited herein, are hereby incorporated by reference in their entirety to the same extent as if each individual publication was specifically and individually indicated to be incorporated by reference.

The terms and expressions used herein have been used as terms of description and not of limitation, and there is no intention in the use of such terms of excluding any equivalents of the features shown and described or portions thereof. It is recognized that various modification are possible within the scope of the invention claimed. Thus, it should be understood that although the present invention has been specifically disclosed by preferred embodiments and other features, modification and variation of the invention embodied therein herein disclosed may be used by those skilled in the art, and that such modification and variations are considered to be within the scope of this invention.

I claim:

1- A UVC germicidal subject hand device comprising:
a. an irradiation chamber;
b. an opening capable for internal hand placement;
c. a micro sensor trigger;
d. an aluminum perforated screen on the inner top surface of the outer box; and
e. at least one UVC germicidal lamp within the aluminum perforated screen wherein up to 1500 vrms is needed to start the lamp and 900 vrms is needed to run the lamp in order to kill 99.999% of all germs within the device
wherein placement of subject's hands into the irradiation chamber prevents cross-contamination of microbes from hand contact.

2- The device of claim 1 having LED indicator lights on the front face of the outer box to indicate power and illumination.

3- The device of claim 1 having dual micro sensor triggers for synchronizing the movement of the user's hands with the on or off status of the UVC lamps.

4- The device of claim 1 having a metal pocket on the outside surface of the bottom of the outer box for a 12 volt poser connection and a cash drawer POS interface.

5- The device of claim 1 further having a microprocessor to control transformers, potentiometer and LEDs that act together to illuminate, monitor and report the status of the illumination process.

6- The device in claim 3 having two separate digital micro switches to display illumination times.

7- The device in claim 1 having an aluminum perforated screen with a 45 degree angle edge on the front and rear portions to provide a protective enclosure for the lamp.

8- The device in claim 1 having an aluminum perforated screen with an inner surface painted black to prevent reflection of emitted UV light.

9- The device in claim 1 having two 5 watt UVC germicidal lamps with a wavelength of 253.7 nm with 2600 microwatts at 1 inch per cm².

10- The device of claim 1 having a WIFI network for interfacing with a computer or mobile device to provide information on UVC bulb, circuit and system power status.

11- The device of claim 1 further having a kiosk comprising:
a. A sensor for detecting body temperature in a person, and
b. An on-screen video to provide a source of entertainment for the user while their hands are sanitized.

12- An anti-germicidal method for sanitizing subject's hands in a retail store comprising:
a. interfacing a germicidal device of claim 1 with a sanitizing station;
b. inserting a defined portion of the subject's hand into an opening in the device;
c. irradiating the subject's hands; and
d. removing the subject's hands from the device.

13- A device for curing the surface of coated nails on a subject comprising:
a. an irradiation chamber;
b. an opening capable for internal placement of the subject's nails coated with a nail enhancement product;
c. a micro sensor trigger;
d. an aluminum perforated screen on the inner top surface of the outer box; and
e. at least one UV-A lamp emitting around 350 nm within the aluminum perforated screen wherein up to 1500 vrms is needed to start the lamp and 900 vrms is needed to run the lamp in order to cure the nail paint
wherein placement of subject's nails into the irradiation chamber cures the nail enhancement product on the nail surface.
